(19) United States
(12) Patent Application Publication
(10) Pub. No.: US 2019/0355274 A1
(43) Pub. Date: Nov. 21, 2019

(54) METHOD OF DEVELOPMENT OF CONCENTRATIONS AND DEVICE PRK-1U FOR ITS IMPLEMENTATION

(71) Applicant: Grigorii Petrovich Grabovoi, Belgrade (RS)

(72) Inventor: Grigorii Petrovich Grabovoi, Belgrade (RS)

(21) Appl. No.: 16/415,955

(22) Filed: May 17, 2019

Related U.S. Application Data
(60) Provisional application No. 62/673,151, filed on May 18, 2018.

(51) Int. Cl. G09B 19/00; A61B 5/04; G09B 1/00; G01R 29/08

(52) U.S. Cl. CPC G09B 19/00; G01R 29/0885; G09B 1/00; A61B 5/04005

(57) ABSTRACT

Devices and methods for development of concentration are described herein. A device for development of concentration may include an optical sensing unit. The optical sensing unit may include a plurality of sensitive elements. The plurality of sensitive elements may be configured to sense a signal provided by a user. The signal may be associated with a plurality of electromagnetic fields. The plurality of sensitive elements may be configured to impose, based on the signal, the plurality of electromagnetic fields onto each other to obtain an outgoing signal. The device for development of concentration may further include an optical emitting unit configured to emit the outgoing signal and one or more lenses for focusing concentration of the user. The one or more lenses may be associated with the optical sensing unit.

### CROSS-REFERENCE TO RELATED APPLICATIONS

This application claims priority of U.S. Provisional Patent Application No. 62/673,151 filed on May 18, 2018, entitled “METHOD OF DEVELOPMENT OF CONCENTRATIONS AND DEVICE PRK-1U FOR ITS IMPLEMENTATION”, which is incorporated herein by reference in its entirety.

### TECHNICAL FIELD

The present disclosure relates generally to optical devices and, more specifically, to devices and methods for developing concentration.

### BACKGROUND

The approaches described in this section could be pursued but are not necessarily approaches that have previously been conceived or pursued. Therefore, unless otherwise indicated, it should not be assumed that any of the approaches described in this section qualify as prior art merely by virtue of their inclusion in this section.

It is generally known that cells of a human body, e.g., neurons, produce electrical activity. In particular, nerve impulses generated by neurons are electrical signals that create electromagnetic fields of the human body. Furthermore, some fluids of the human body are known to act as electrolytes and the flow of such fluids may generate fluctuating electromagnetic fields in the human body. However, conventional electromagnetic sensors are not intended for detecting the electromagnetic fields of the human body and are unable to transform electromagnetic signals emitted by the human body.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Provided are devices and methods for development of concentration. In some example embodiments, a device for development of concentration may include an optical sensing unit. The optical sensing unit may include a plurality of sensitive elements. The plurality of sensitive elements may be configured to sense a signal provided by a user. The signal may be associated with a plurality of electromagnetic fields. The plurality of sensitive elements may be configured to impose, based on the signal, the plurality of electromagnetic fields onto each other to obtain an outgoing signal. The device for development of concentration may further include an optical emitting unit configured to emit the outgoing signal and one or more lenses for focusing concentration of the user. The one or more lenses may be associated with the optical sensing unit.

A method for development of concentration may commence with providing one or more lenses for focusing concentration of a user. The one or more lenses may be associated with an optical sensing unit. The method may further include sensing, by a plurality of sensitive elements of the optical sensing unit, a signal provided by the user. The signal may be associated with a plurality of electromagnetic fields. The method may continue with imposing, by the optical sensing unit, based on the signal, the plurality of electromagnetic fields onto each other to obtain an outgoing signal. The method may further include emitting, by an optical emitting unit, the outgoing signal.

Additional objects, advantages, and novel features will be set forth in part in the detailed description section of this disclosure, which follows, and in part will become apparent to those skilled in the art upon examination of this specification and the accompanying drawings or may be learned by production or operation of the example embodiments. The objects and advantages of the concepts may be realized and attained by means of the methodologies, instrumentalities, and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and in which:

FIG. 1 illustrates a general perspective view of a device for development of concentration, in accordance with an example embodiment.

FIG. 2 is a block diagram showing various modules of a device for development of concentration, in accordance with an example embodiment.

FIG. 3 is a schematic diagram illustrating development of concentration of a user using a device for development of concentration, in accordance with an example embodiment.

FIG. 4 is a flow chart illustrating a method for development of concentration, in accordance with an example embodiment.

FIG. 5 is a schematic diagram illustrating a device for development of concentration, according to an example embodiment, in accordance with an example embodiment.

FIG. 6 is a schematic illustration showing elements of a device for development of concentration, according to an example embodiment.

FIG. 7 shows a computing system that can be used to implement a method for development of concentration, according to an example embodiment.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents. In this document, the terms “a” and “an” are used, as is common in patent documents, to include one or more than one. In this document, the term “or” is used to refer to a nonexclusive “or,” such that “A or B” includes “A but not B,” “B but not A,” and “A and B,” unless otherwise indicated.

The present disclosure relates to methods and devices for development of concentration. Specifically, the development of concentration is provided by a device for development of concentration, also referred herein to as a device PRK-1U for development of concentration. The device may include an optical sensing unit configured to sense signals emitted by a user and an optical emitting unit configured to emit an outgoing signal. The device further includes lenses for focusing concentration of the user.

The device may include a housing in which elements of the device may be located. The housing may have a parallelepiped shape. The housing may be provided with a cover placed onto the housing to enclose the elements of the device inside the housing. The lenses for focusing concentration may be attached to an outer surface of the housing or to the cover. The user may be located in proximity to the device. The development of concentration of the user may be provided by focusing user attention on a receiver of the device and controlling the results of the concentration. The lenses may be configured to be the receiver of concentration of the user. To initiate development of concentration, the user may start concentrating on the lenses. Specifically, the user may focus user attention on the lenses and direct thoughts to the lenses of the device. The concentration of the user may include thoughts related to providing an eternal life, including concentration on being healthy, concentration on having the quality of control forecasting or control foresight, concentration on rejuvenation, and so forth.

As known in psychology, the stronger a person concentrates on a goal, the better events in life of the person are optimized and the faster the goal is achieved. When concentrating, the user may perform the following actions. The user may imagine user consciousness as a sphere around the user body informationally supported by the user body itself. The further action of the user may include imagining that the sphere transforms into the shape similar to the shape of the user body and then superimposes onto the surface of the user body. At the moment of superimposing, the user may imagine that the inner surface of the body-like shape comes into contact with the surface of the user body and that the radiation from the outer surface of this body-like shape spreads to all external infinite space relative to the user body. The infinite space is considered to be the eternal reality connected with the organism of the user, which results in development of the concentration on eternal life.

The devices and methods described herein are based on the principle of similarity. The principle of similarity is based on the theory of wave synthesis in combination with the unified reality theory (see Ph.D. Thesis in Physical and Mathematical Sciences, G. P. Grabovoi, “Research and Analysis of Fundamental Definitions of Optical Systems for Prediction of Industrial Nature Earthquakes and Disasters”, Moscow, RAEN Publishing House, 1999, pp. 9-19; patent of the inventor No. RU 2148845C1 titled “Method of Prevention of Catastrophes and Equipment for its Realization”; patent of the inventor No. RU 2163419C1 titled “Data Transmission System,” which are incorporated herein by reference in their entirety). The devices and methods are further based on physical and mathematical theory, experimental results, physical and mathematical calculations, and the results of these calculations set forth in the publication titled “Research and Analysis of the Fundamental Definitions of Optical Systems in Disaster Prevention and Predictive Microprocessor Control”, “Electronic Equipment, Series 3, Microelectronics”, 1999, edition 1 (153), and other scientific materials.

In accordance with the wave synthesis theory, reality can be considered as a periodic intersection of stationary regions with dynamic regions, while in the intersection zones a synthesis of a dynamic wave and a stationary wave occurs. Any reality phenomenon can be defined in form of optical systems. Human perception is performed using image-bearing elements of light that contain information. In case of transmitting information from a person generating information to be transmitted to an optical sensing element, the person may be considered to be a transmitting optical system. The transmitted information generated by thoughts of the person is received by an optical sensing unit to which the person directs the generated thought. As a thought is an electromagnetic wave, it can be transmitted as an element of optical system. Sensitive elements of the optical sensing unit preferably have the shape of a sphere, as the spherical shape of the sensitive element provides the maximum activation of the sensitive element due to internal reflection of signals. The collection of trial records and testimonies of use of device PRK-1U for the development of the concentration is presented in the Appendix of Specification.

The device for development of concentration performs the imposition of fields from the generation of biological signals and electromagnetic fields (electromagnetic waves generated by the user) according to the principle of universal connection with control of the purpose of concentration. The device further develops concentration of creational control.

In the wave synthesis theory, it is known that a thought generated in a form of radiation simultaneously has two quantum states. The first state is located on a sensing element of a signal transmitter, and the second state is located on a signal receiver. Based on these principles, the device for interacting with thoughts to develop the concentration as described herein was created.

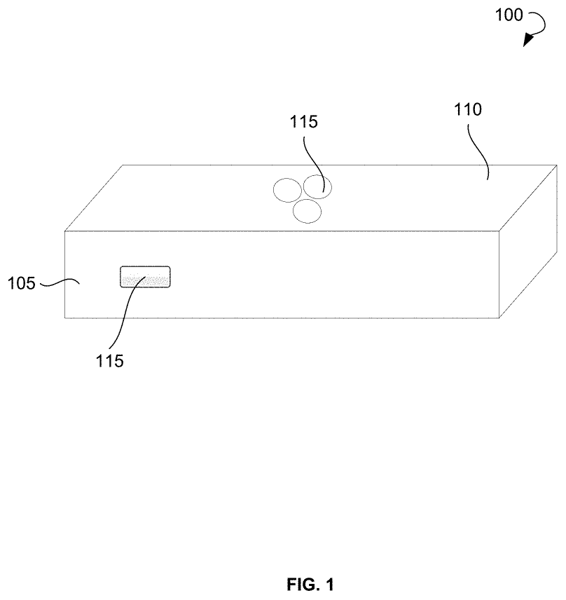

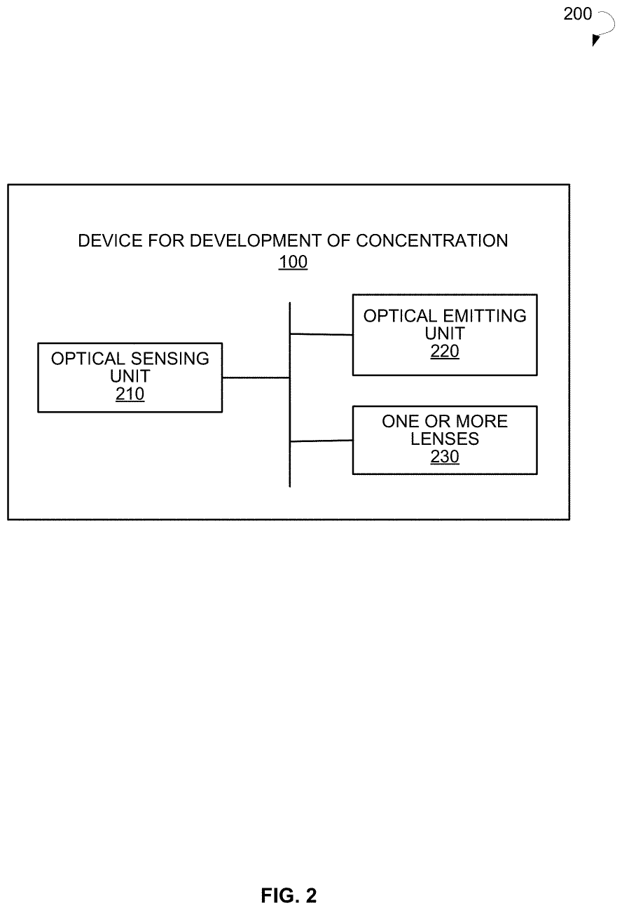

Referring now to the drawings, FIG. 1 is a general perspective view of a device 100 for development of concentration. The device 100 may include a housing 105 and a cover 110. In an example embodiment, the housing 100 may include a box of a rectangular shape. The device may further have a switch 115 to turn on and turn off the device 100. The device 100 may further include lenses 120. The lenses 120 may be attached to an outer surface of the cover 110. In an example embodiment, the lenses 120 may be made of glass. Further elements of the device 100 are shown in detail with reference to FIGS. 2-6.

FIG. 2 is a block diagram showing various units of a device 100 for development of concentration, in accordance with certain embodiments. Specifically, the device 100 may include an optical sensing unit 210, and optical emitting unit 220, and one or more lenses 230. The one or more lenses 230 for focusing concentration of a user may be associated with the optical sensing unit. The device 100 may further include a housing and a cover. The one or more lenses 230 may be disposed on the cover.

The optical sensing unit 210 may have a plurality of sensitive elements. In an example embodiment, the plurality of sensitive elements may be spherical. In an example embodiment, the sensitive elements may be made of glass. The plurality of sensitive elements may be configured to sense a signal provided by the user. The signal may be associated with a plurality of electromagnetic fields. The signal provided by the user may be a biological signal. The biological signal may include an electromagnetic wave associated with thoughts generated by the user when concentrating on the one or more lenses 230 for focusing concentration. Specifically, the information (signal) may be generated in form of electromagnetic radiation by the user. The user concentrates the electromagnetic radiation created by thought on the lenses located on the upper surface of the device.

The plurality of sensitive elements may be further configured to impose, based on the signal, the plurality of electromagnetic fields onto each other to obtain an outgoing signal. The optical emitting unit may be configured to emit the outgoing signal. In an example embodiment, the optical emitting unit 220 may include an optical lens. In an example embodiment, the optical lens may be made of glass. The optical emitting unit 220 may emit the outgoing signal in a form of at least an optical signal. In an example embodiment, the device 100 may include a further plurality of sensitive elements. The further plurality of sensitive elements may include crystals.

In an example embodiment, the device 100 may further include a converting unit configured to convert the outgoing signal into an electrical signal. In an example embodiment the converting unit may be connected to a processing unit. The processing unit may be in communication with the optical sensing unit and the optical emitting unit and perform processing of sensed signals, imposed signals, optical signals, and outgoing signals. The device 100 may further include a power source in communication with the optical sensing unit 210 and the optical emitting unit 220.

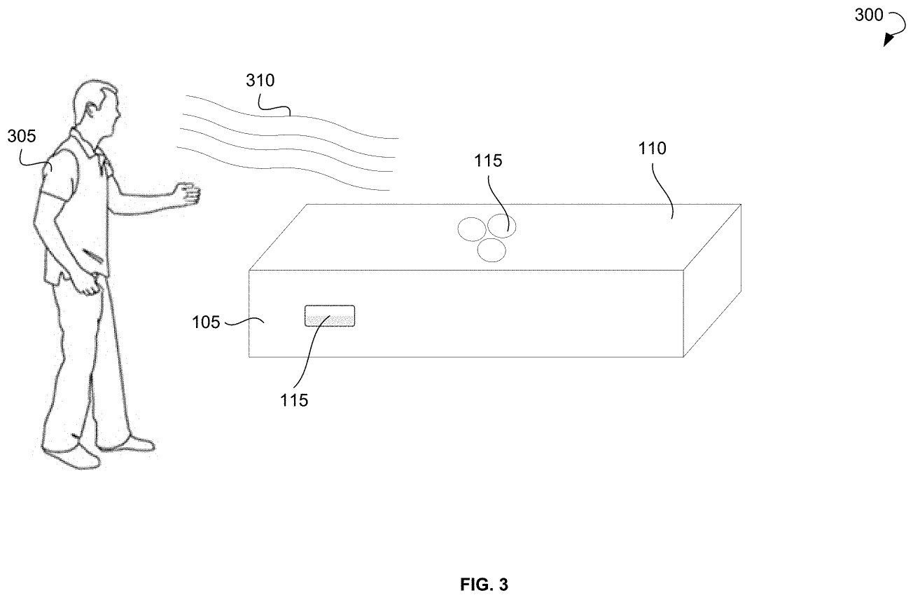

FIG. 3 is a schematic diagram 300 illustrating development of concentration of a user using a device for development of concentration, according to an example embodiment. A user 305 may be located in a proximity of the device 100 for development of concentration. The user 305 may concentrate user attention on lenses 115 of the device 100. By concentrating, the user 305 generates thoughts which are electromagnetic signals 310. The thoughts may contain the purpose of concentration, such as concentration on eternal life, concentration on being healthy, concentration on having the quality of control forecasting or control foresight, concentration on rejuvenation, and so forth. The action of concentration for the current time and future time may be performed with respect to a sensing element of the optical emitting unit consisting of lenses. The user 305 may perform circular movements associated with the concentrations (i.e., direct thoughts) by following a direction from a lens of a smaller diameter counterclockwise to lenses of a larger diameter. In the case of concentrations related to the current time and future time, a concentration beam may be directed in a direction from an outside of the device 100 to an inner space of the device 100.

If the concentration of the user 305 relates to past events, the user 305 may perform circular movements associated with the concentrations by following a direction from a lens of a smaller diameter clockwise to lenses of a larger diameter. The concentration beam may be directed in a direction from an inside the device 100 to an outside space.

In accordance with the information transmission on the basis of the wave synthesis theory, another quantum state of the thoughts may be projected on a signal receiver in a form of an optical emitting unit located inside the device 100.

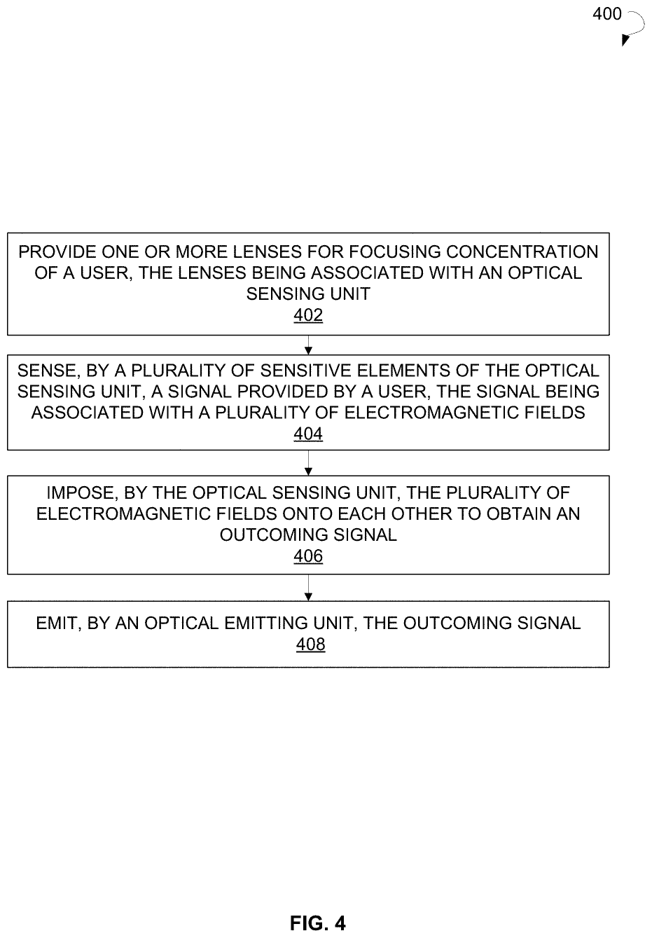

FIG. 4 is a process flow diagram showing a method 400 for development of concentration, according to an example embodiment. In some embodiments, the operations may be combined, performed in parallel, or performed in a different order. The method 400 may also include additional or fewer operations than those illustrated.

The method 400 may commence with providing one or more lenses for focusing concentration of a user at operation 402. The lenses may be associated with an optical sensing unit. The method 400 may further include sensing, by a plurality of sensitive elements of the optical sensing unit, a signal provided by the user at operation 404. The signal may be associated with a plurality of electromagnetic fields. The plurality of sensitive elements may be spherical. The signal provided by the user may be a biological signal.

The method 400 may further include imposing, based on the signal, by the optical sensing unit, the plurality of electromagnetic fields onto each other to obtain an outgoing signal at operation 406. Specifically, the method 400 may be performed by using signal conditioning by imposing electromagnetic fields resulting from the generation of a biological signal to each other. The method 400 may be performed in accordance with the principle of universal connection with control of the purpose of concentration.

The method 400 may further include emitting, by an optical emitting unit, the outgoing signal. The optical emitting unit may include an optical lens. The optical emitting unit may emit the outgoing signal in the form of at least an optical signal. The method 400 may further include converting, by a converting unit, the outgoing signal into an electrical signal.

In an example embodiment, the method 400 may further include providing a power source. The power source may be in communication with the optical sensing unit and the optical emitting unit. In an example embodiment, the method 400 may further include providing a housing and a cover. The one or more lenses may be disposed on the cover.

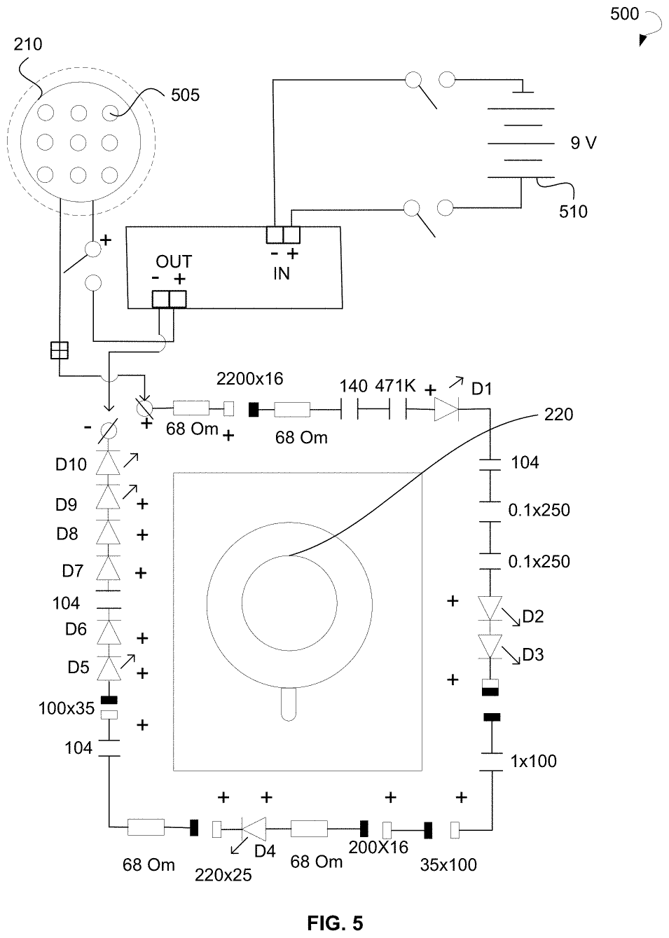

FIG. 5 is a schematic diagram 500 illustrating a device for development of concentration, according to an example embodiment. The elements shown on FIG. 5 may be located inside a housing of the device for development of concentration. The device for development of concentration may include an optical sensing unit 210. The optical sensing unit 210 may include a plurality of sensitive elements 505. The optical sensing unit 210 may be in communication with an optical emitting unit 220. The device for development of concentration may further include a power source 510.

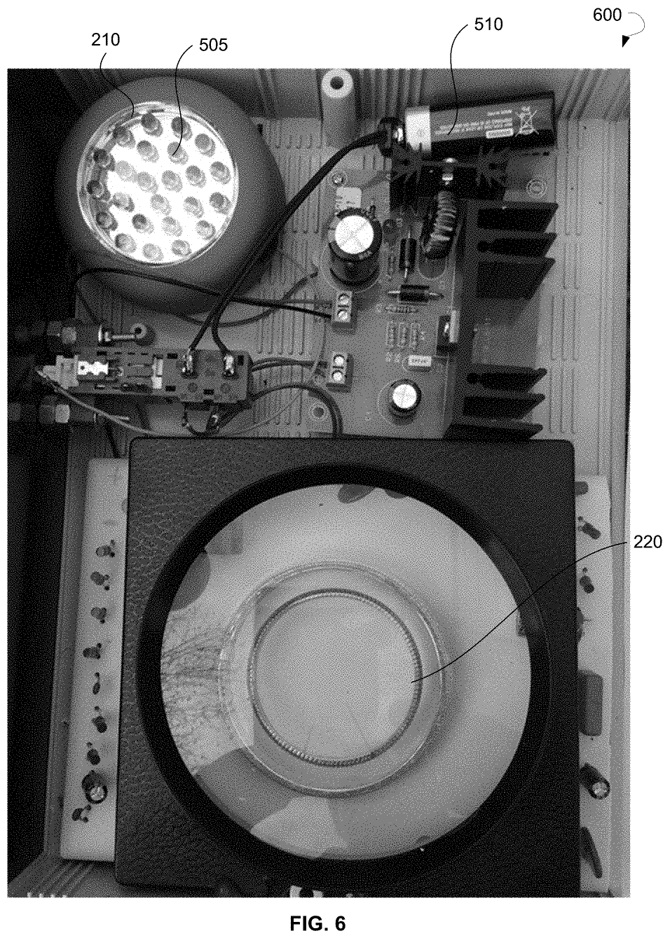

FIG. 6 is a schematic illustration 600 showing elements of a device for development of concentration, according to an example embodiment. The device for development of concentration may include an optical sensing unit 210, a plurality of sensitive elements 505, an optical emitting unit 220, and a power source 510.

In an example embodiment, figures may be placed in a proximity of lenses of the optical emitting unit 220. For example, figures 1, 4, 5 may be placed (e.g., written) near a smaller lens, and figures 2, 7, 8, and 9, 0, 6, 3 may be placed near larger lenses. The development of concentrations in the case of the presence of figures near the lenses can be made by concentrating the lenses on a cover of the device in a way described above. Additionally, the concentration on the figures may be added.

In a further example embodiment, the device for development of concentration may have concentration enhancement elements. For example, the concentration enhancement elements may include the elements of the optical emitting unit. The concentration enhancement elements may include crystals, e.g., diamonds or rock crystals.

In an example embodiment, the device for development of concentration may be used through video monitoring of the device by a user. The device for development of concentration is applicable in various fields related to providing eternal life, such as becoming healthy, developing the quality of control forecasting or control foresight, rejuvenation of organism, and so forth.

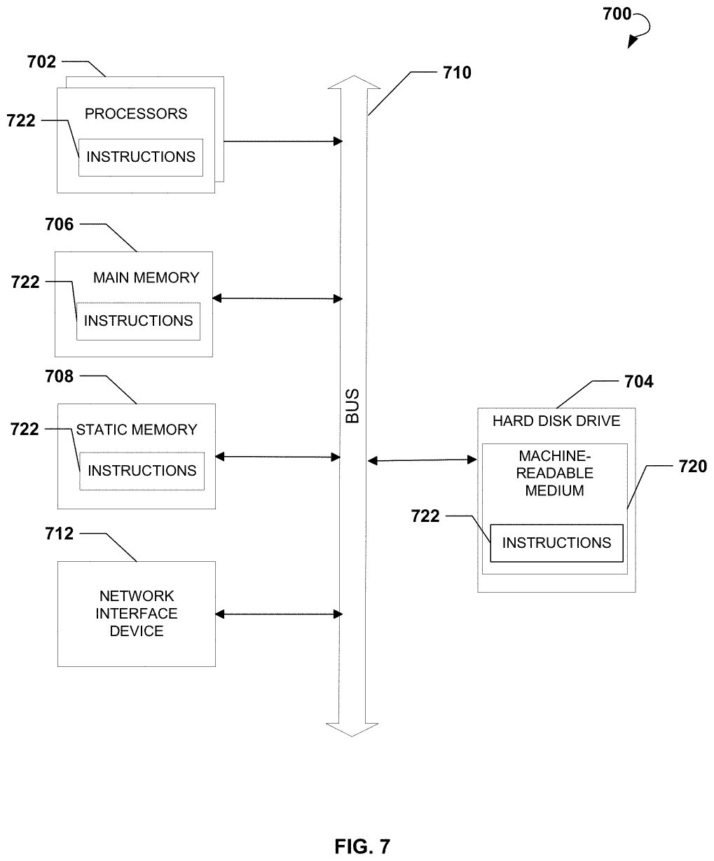

FIG. 7 shows a diagrammatic representation of a computing device for a machine in the exemplary electronic form of a computer system 700, within which a set of instructions for causing the machine to perform any one or more of the methodologies discussed herein can be executed. In various exemplary embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a PC, a tablet PC, a set-top box, a cellular telephone, a digital camera, a portable music player (e.g., a portable hard drive audio device, such as an Moving Picture Experts Group Audio Layer 3 (MP3) player), a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 700 may include a processor or multiple processors 702, a hard disk drive 704, a main memory 706 and a static memory 708, which communicate with each other via a bus 710. The computer system 700 may also include a network interface device 712. The hard disk drive 704 may include a computer-readable medium 720, which stores one or more sets of instructions 722 embodying or utilized by any one or more of the methodologies or functions described herein. The instructions 722 can also reside, completely or at least partially, within the main memory 706 and/or within the processors 702 during execution thereof by the computer system 700. The main memory 706 and the processors 702 also constitute machine-readable media.

While the computer-readable medium 720 is shown in an exemplary embodiment to be a single medium, the term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media. Such media can also include, without limitation, hard disks, floppy disks, NAND or NOR flash memory, digital video disks, Random Access Memory, Read-Only Memory, and the like.

The example embodiments described herein may be implemented in an operating environment comprising software installed on a computer, in hardware, or in a combination of software and hardware.

Thus, devices and methods for development of concentration are described. Although embodiments have been described with reference to specific exemplary embodiments, it will be evident that various modifications and changes can be made to these exemplary embodiments without departing from the broader spirit and scope of the present application. Accordingly, the specification and drawings are to be regarded in an illustrative rather than a restrictive sense.

What is claimed is:

1. A device for development of concentration, the device comprising:
an optical sensing unit, the optical sensing unit comprising a plurality of sensitive elements, wherein the plurality of sensitive elements are configured to:
sense a signal provided by a user, the signal being associated with a plurality of electromagnetic fields; and
based on the signal, impose the plurality of electromagnetic fields onto each other to obtain an outgoing signal;
an optical emitting unit configured to emit the outgoing signal; and
one or more lenses for focusing concentration of the user, the one or more lenses being associated with the optical sensing unit.

2. The device of claim 1, further comprising a power source in communication with the optical sensing unit and the optical emitting unit.

3. The device of claim 1, wherein the plurality of sensitive elements are spherical.

4. The device of claim 1, wherein the signal provided by the user is a biological signal.

5. The device of claim 1, further comprising a housing and a cover.

6. The device of claim 5, wherein the one or more lenses are disposed on the cover.

7. The device of claim 1, wherein the optical emitting unit includes an optical lens.

8. The device of claim 1, wherein the optical emitting unit emits the outgoing signal in form of at least an optical signal.

9. The device of claim 1, further comprising a further plurality of sensitive elements, wherein the further plurality of sensitive elements includes crystals.

10. The device of claim 1, further comprising a converting unit configured to convert the outgoing signal into an electrical signal.

11. A method for development of concentration, the method comprising:
providing one or more lenses for focusing concentration of a user, the one or more lenses being associated with an optical sensing unit;
sensing, by a plurality of sensitive elements of the optical sensing unit, a signal provided by the user, the signal being associated with a plurality of electromagnetic fields;
based on the signal, imposing, by the optical sensing unit, the plurality of electromagnetic fields onto each other to obtain an outgoing signal; and
emitting, by an optical emitting unit, the outgoing signal.

12. The method of claim 11, further comprising providing a power source, wherein the power source is in communication with the optical sensing unit and the optical emitting unit.

13. The method of claim 11, wherein the plurality of sensitive elements are spherical.

14. The method of claim 11, wherein the signal provided by the user is a biological signal.

15. The method of claim 11, further comprising providing a housing and a cover.

16. The method of claim 15, wherein the one or more lenses are disposed on the cover.

17. The method of claim 11, wherein the optical emitting unit includes an optical lens.

18. The method of claim 11, wherein the optical emitting unit emits the outgoing signal in form of at least an optical signal.

19. The method of claim 11, further comprising converting, by a converting unit, the outgoing signal into an electrical signal.

20. A device for development of concentration, the device comprising:
an optical sensing unit, the optical sensing unit comprising a plurality of sensitive elements, wherein the plurality of sensitive elements are configured to:
sense a signal provided by a user, the signal being associated with a plurality of electromagnetic fields, wherein the signal provided by the user is a biological signal; and
based on the signal, impose the plurality of electromagnetic fields onto each other to obtain an outgoing signal;
an optical emitting unit configured to emit the outgoing signal, wherein the optical emitting unit emits the outgoing signal in form of at least an optical signal;
one or more lenses for focusing concentration of a user, the one or more lenses being associated with the optical sensing unit; and
a converting unit configured to convert the outgoing signal into an electrical signal.
